(19) United States
(12) Patent Application Publication
(10) Pub. No.: US 2004/0120983 A1
(43) Pub. Date: Jun. 24, 2004

(54) Nutritional supplement

(76) Inventor: Philip Connolly, Lake Arrowhead, CA (US)

Correspondence Address: Paul M. Denk, 763 South New Ballas Road, St. Louis, MO, 63141, US

(21) Appl. No.: 10/326,946

(22) Filed: Dec. 23, 2002

(51) Int. Cl. A61K 31/7012; A61K 31/198; A61K 31/70; A61K 31/715; A61K 31/185

(52) U.S. Cl. 424/439; 514/23; 514/54; 514/561; 514/562; 514/553; 514/565; 514/563; 514/53

(57) ABSTRACT

A nutritional supplement for sports nutrition and also as a general population dietary supplement for aging people is provided. The nutritional supplement is a combination of a high glycemic sugar or carbohydrate, vitamin B6, electrolytic minerals, and amino acids some of which have neuromodulating properties and some of which are muscle cell volumizers. The amino acids included in the nutritional supplement include the amino acids include glutamine, taurine, creatine, cysteine, glycine, leucine, and arginine. The amino acids having neuromodulating properties is chosen from the group consisting of glutamine, taurine, glycine, and cysteine, and combinations thereof. The amino acids having cell volumizing properties is chosen from the group consisting of creatine, taurine, and glycine, and combinations thereof. The combination of the ingredients has a synergistic effect of increasing muscle mass, increasing physical abilities (strength, stamina, and endurance), and decreasing body fat.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

This invention relates to a nutritional or dietary supplement which stimulates metabolism to an anabolic state to increase skeletal muscle mass and reduce body fat content.

The sports nutrition industry encompasses a range of products including protein powders, supplemented amino acids, hormones, and products known as pro-hormones, hormone mimicking chemicals, that might differ only slightly in structure from the hormones that they are supposed to mimic. All of these products are designed to promote extra muscle tissue growth (anabolism). The methods for achieving extra muscle tissue growth vary by the agent involved. However, each and every agent utilized in sports nutrition will increase muscle tissue growth by either directly triggering muscle protein synthesis, as in the case of hormones and prohormones, triggering production of anabolic hormones in the body, or decreasing the time necessary for the body to repair and rebuild muscle tissue after exercising (decreased recovery times). In all cases the goal of sports nutrition supplements is to build more muscle tissue faster and to prevent injuries.

The most common form of dietary supplementation in the sports nutrition industry is protein powder. Virtually every athlete and want-to-be athlete supplements their daily diet with extra protein in the form of powders or ready-to-drink protein shakes. Protein is metabolized by the body to its building blocks—amino acids. The amino acids can then be used by the body to synthesize needed proteins and anabolic hormones for synthesis of new muscle tissue. These protein powders, however, are not sufficient in most cases to produce the large, muscular athletic bodies seen today. Consequently, many serious athletes will supplement their normal/protein shake diet with additional assistance—hormones, pro-hormones, and amino acids.

Use of amino acid supplementation is very common among serious athletes. Many consumers will supplement their dietary regime with glutamine and creatine, as well as other amino acids. Amino acid supplementation, however, is not thought to be much more effective than protein supplementation in helping an athlete to achieve real lean tissue gains. Very few dedicated athletes report significant benefits from amino acid supplementation. Despite numerous written reports of controlled clinical trials showing the benefits of amino acid supplementation, the effects are not as pronounced under real-life dietary/training conditions as they are in controlled clinical studies. Therefore, amino acid supplementation is not considered to be a viable option for gaining muscle mass.

Use of hormones (examples: Testosterone, Growth Hormones) have been banned by most international amateur and professional athletic organizations. Athletes that test positive for use of hormones to augment their natural physical abilities are usually suspended or banned from competition. Consequently, hormones are not considered to be an attractive, legal, or healthy option for serious athletes.

In the past 10 years, the use of pro-hormones has risen significantly. Pro-hormones were originally developed to circumvent testing methods for the banned hormones. As an example, androstenadiole (Andro) was originally developed in Eastern Europe during the cold war to assist athletes from Iron Curtain countries to gain extra muscle mass while allowing them to pass Olympic drug testing procedures for banned substances. Andro has a steroid-like effect on the metabolism, causing the body to produce more muscle tissue than it would otherwise produce and, therefore, is significantly anabolic, like the banned steroids and growth hormones. Olympic drug testers, however, had no idea of the existence of Andro and, therefore, had no method for testing or detecting its use by an athlete. Eastern European athletes used Andro for years with impunity. Only recently, after the fall of the Iron Curtain did the full story of Andro come to light. Athletes in the USA started to use Andro. However, with an increase in the use of Andro (and other pro-hormones like it) came a move to ban these pro-hormones in sports. Each year a new list of banned pro-hormones is published by the International Olympic Committee and other professional and amateur sports organizations, and each year new pro-hormone products are developed to replace the banned substances. Use of pro-hormones is simply a short term method for cheating organized sports testing methods and is not a long term option for athletic performance or the health of the athlete. Pro-hormones are agents that possess chemical structures very close to anabolic hormones, such as the steroidal compounds or growth hormones. The purpose of a pro-hormone is to provide an anabolic reaction in the body similar to anabolic steroids without putting the athlete in danger of failing a banned substance drug test. Since nobody can predict the long term health risks of prolonged use of pro-hormones, sports organizations and legislatures worldwide continue to ban pro-hormones as they increase in use. Consequently, pro-hormone supplementation is not a desirable method for gaining increased muscle mass as one cannot rely on being able to use pro-hormones in future years and, from a medical viewpoint, they are probably not healthy alternatives.

Numerous companies have concentrated on developing safe supplements that will augment muscle tissue growth and which do not contain banned substances or substances that are likely to be banned from sports in the future. To date, most of the products have contained one or two active ingredients and have not been as successful at forming muscle tissue as expected.

### BRIEF SUMMARY OF THE INVENTION

We have developed a new product that can be used as a supplement for sports nutrition and also as a general population dietary supplement for aging people. The product is a combination of a high glycemic sugar or carbohydrate, vitamin B6, electrolytic minerals, and amino acids some of which have neuromodulating properties and some of which are muscle cell volumizers. The amino acids included in the nutritional supplement include the amino acids include glutamine, taurine, creatine, cysteine, glycine, leucine, and arginine. The amino acids having neuromodulating properties is chosen from the group consisting of glutamine, taurine, glycine, and cysteine, and combinations thereof. The amino acids having cell volumizing properties is chosen from the group consisting of creatine, taurine, and glycine, and combinations thereof.

In a preferred blend, the nutritional supplement includes by weight, about 40% to about 65%, glycemic carbohydrate or sugar; about 10% to about 30% Glutamine; about 10% to about 30% Creatine; about 7.1% glycine; about 2.4% taurine; about 2.4% leucine; about 1.2% cysteine; about 0.2% arginine; about 1.4% citrate; about 0.2% Sodium Chloride; about 0.2% Potassium Chloride; about 0.01% to about 0.04% vitamin B6; and about 2.4% Flavor, sweetener, and color. On a per serving (or single dose) basis, the components of the supplement are calculated so that each serving supplies about 23 grams of high glycemic carbohydrate, about 6.66 grams of glutamine, about 5 grams of creatine, about 3 grams of glycine, about 1 gram of taurine, about 1 gram of leucine, about 0.5 grams of cysteine, about 100 mg of arginine, about 60 mg potassium, about 50 mg sodium, and about 200% of the USRDA for Vitamin B6. The pH of the total blend when dispersed into 16 ounces of water was 4.6.

The theory behind the product is that all of the ingredients combine to produce an anabolic (body building) metabolic reaction when the product is consumed. The high glycemic sugar stimulates production of insulin. The manufactured insulin increases absorption of the nutrients through the intestinal walls and helps to transport the amino acids to skeletal muscle tissue cells. Once at the muscle cells, the insulin assists in transporting the amino acids from the bloodstream into the muscle cells. These amino acids are the primary building blocks of skeletal muscle tissue. Once inside the muscle cells, the amino acids also attract water and water soluble nutrients, such as the electrolyte minerals, by osmosis to the muscle cells. The result is larger, more functional skeletal muscle tissue. At the same time, other included gluconeogenic amino acids and the vitamin 136 play a role in maintaining blood sugar levels in the presence of the increased insulin and throughout the remainder of the day so that the cells have sufficient energy to utilize the amino acids for protein synthesis, muscle tissue growth, improving athletic performance, and to help prevent skeletal injury. We have tried to minimize the doses of each ingredients, providing efficacious amounts of each ingredient but not exceeding safe and healthy limits. Due to the synergism between the active ingredients that we have encountered, it is not necessary to exceed safe and healthy limits for any of the ingredients. It is doubtful that any extra benefits could be realized, even if one were to significantly increase usage levels of many of the ingredients in the formula.

### DETAILED DESCRIPTION OF THE INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. Additionally, it is to be understood that the invention is not limited in its application to the details of construction and the arrangements of components set forth in the following description or illustrated in the drawings. The invention is capable of other embodiments and of being practiced or being carried out in various ways. Also, it is to be understood that the phraseology and terminology used herein is for the purpose of description and should not be regarded as limiting.

We have developed a product that would includes a number of anabolic metabolism precursors. During the development of the product, we sought to manufacture a supplement that would assist the body in manufacturing muscle tissue. The mixture comprises only ingredients that are thought to be safe and are common in many other supplements. These ingredients, by themselves, have been shown to have some effect on muscle tissue growth and are considered to be safe and will not be subject to future bans or health risks. However, the mixture, unexpectedly, has a synergistic effect on anabolism when consumed. Subjects that have experimentally used the mixture not only report muscle tissue growth but also report loss of body fat, increased stamina, increased strength, feeling younger, and improved athletic performance.

The mixture comprises a simple, high glycemic sugar with specific quantities of the amino acids Glutamine, Taurine, Creatine, Cysteine, Glycine, Leucine, and Arginine. The mixture also includes electrolytic sodium, potassium, and Vitamin B6. The combination of these ingredients in an optimum ratio creates a synergistic reaction that stimulates the metabolism to an anabolic state.

The high glycemic simple sugar is provided to stimulate a short term, fixed amount of insulin production in the body. Insulin has long been recognized as an anabolic hormone if it is released at the proper time in the body. If insulin is released at the wrong time into the bloodstream, it can be a destructive, or catabolic, hormone. When a high glycemic (insulin producing) sugar is consumed, the body manufactures insulin. The amount of insulin produced is dependent on the quantity and glycemic nature of the carbohydrate consumed. When the body produces insulin, the insulin stimulates the intestines to increase nutrient transport across the intestinal wall and into the blood stream. Insulin also activates cell transporters, bringing them to the cell surface so that they can assist in moving nutrients from the bloodstream into cells. If timed properly, insulin release has been shown to assist in the efficient transport of amino acids from the intestines into muscle cells. If timed poorly, insulin release has been shown to increase the transport of fats into cells for storage, thereby increasing body fat content. It is very important for an athlete desiring to build new skeletal muscle while minimizing body fat content to time the release of insulin properly. Some bodybuilders, for instance, have been known to avoid high glycemic foods in order to minimize their body's production of insulin, and then inject themselves with insulin at specific times of the day to take advantage of properly timed insulin release. Due to the difficulty in controlling insulin release and the associated risks of increasing stored fat, many athletes including, but not limited to, bodybuilders, have chosen to avoid high glycemic foods and limit their overall insulin production. While limiting insulin production can effectively help to maintain a low body fat content, the anabolic effects of insulin amino acid transport to muscle cells will also be limited or non-existent, making it more difficult for an athlete to add new skeletal muscle. We chose to include an insulin stimulating carbohydrate in our formula so that the body would manufacture sufficient insulin to help transport amino acids and other nutrients to muscle cells for a 60 to 90 minute time period after consumption. Such sugars would come from the family of high glycemic carbohydrates of sucrose, glucose, dextrose, fructose, and some complex carbohydrates such as white flour, potato starch, rice starch, etc.

The amino acids included in the mixture have all been shown individually to have significant effects on anabolism, muscle tissue growth and athletic performance. Creatine is synthesized in the body from the amino acids glycine and arginine. Creatine can be found concentrated in muscle tissue and has been shown to be crucial in regulating energy flow within skeletal muscle tissue. Creatine also increases a muscle cell volume by attracting water to muscle cells. Carefully controlled clinical studies have demonstrated that daily dietary supplementation of 5 gram doses of creatine can result in increased skeletal muscle mass. However, in actual daily life practice, supplementation with creatine does not work as effectively. Consequently, many manufacturers have formulated creatine containing products with creatine contents well in excess of the clinically recommended 5 gram per day doses, with some products containing as high as 50 grams of creatine per dose. There is no scientific evidence to show that increased doses of creatine will result in increased muscle growth, but many athletes consume large doses of creatine every day in an effort to obtain the desired effect from the creatine. In recent years, there have been some negative papers written concerning creatine supplementation, many of them dealing with the larger than recommended daily doses. Creatine draws water to skeletal muscle tissue, and can take that necessary moisture away from other skeletal tissue, such as tendons, ligaments, and bone. Some investigators have suggested that supplementation with larger doses of creatine every day can result in “dry” bones, tendons, and ligaments and increase the risk of injury from broken bones and/or torn ligaments. The mixture preferably uses the clinically proven dose of 5 grams of creatine to avoid the extra risk of “drying” out bones and connective tissues while maintaining the benefit of creatine. The creatine can be provided as creatine monohydrate, buffered creatine, N-acetyl-creatine, creatine phosphate, creatine citrate, creatine glycerol phosphate, or any other source of creatine.

Glutamine is strongly concentrated in cells in the central nervous system, intestines, and skeletal muscle. Glutamine has been shown to play an important role in maintaining intestinal health (improving nutrient absorption), formation of skeletal muscle tissue, and regulating muscle protein turnover. Glutamine is the most common amino acid found in skeletal muscle tissue. Glutamine is also an important precursor of glutathione, the body's own free radical scavenger. Studies have shown that increased levels of intracellular glutathione lead to increased skeletal muscle mass. Recent studies have shown that persons under stress (this would include hard training athletes) demonstrate depletion of glutamine and that supplementation with up to 20 grams of glutamine per day will improve metabolic performance and muscle tissue growth. A recent investigator reported that doses of from 2 to 10 grams of glutamine can result in as much as 15% extra skeletal muscle mass. One can either use L-glutamine or peptide bonded glutamine. Peptide bonded glutamine is preferred as the glutamine source because studies have shown that peptide bound amino acids are more efficiently absorbed across the intestinal wall than are free amino acids (i.e., L-glutamine).

Taurine is a sulfur containing amino acid found primarily in meat but that can also be manufactured by the body from cysteine. Like creatine and glutamine, taurine is found primarily concentrated in skeletal muscle tissue and is the second most common amino acid found in skeletal muscle tissue. Taurine, like, creatine increases muscle cell volume by boosting cell hydration. Investigators recently have claimed that taurine may also assist in stimulating protein synthesis. Other investigators have claimed that taurine can enhance amino acid transport into muscle cells. The exact function of taurine is not fully understood at this time, but it is known that human maternal milk contains substantial quantities of glutamine, creatine, and taurine as free amino acids. It is thought that these three amino acids play an important part in postnatal development, tissue repair, and growth in infant human beings. The first few years of life are of interest in sports nutrition. The first two years in a human's life are a significant growth and anabolic time, with most humans growing approximately half of the cell numbers that their bodies will have when they are adults. There is no other stage in human life where the cell growth is as explosive. Since a primary principle in sports nutrition is to increase cell growth so that the athlete can generate muscle tissue faster, one should look at the nutrients that are prevalent in the postnatal diet of human beings so that one can accelerate cell growth in the adult human. All three amino acids are present in the mixture because they appear to play an important role in generating and repairing skeletal muscle tissue.

Glycine is not considered to be an essential amino acid because it can be synthesized in the body from either threonine or serine. Glycine, however, has been shown to serve a number of important functions in the body. Glycine, like creatine and taurine, draws water to muscle cells and is a muscle cell volumizer due to the hydration function. Glycine, along with glutamine and cysteine, is a precursor of the very important glutathione production in the body. Glycine plays an important role in the control of gluconeogenesis, or the manufacture of blood sugar from amino acids in the liver, and prevention of hypoglycemia. Glycine, then, becomes important in a sports nutrition product where we are trying to stimulate insulin production. The manufactured insulin has the potential to decrease blood sugar and, therefore, body energy levels. Glycine plays a regulatory role in helping to maintain constant blood sugar levels. In higher doses, glycine has been shown to stimulate the release of growth hormone by the body—another anabolic benefit. Glycine is also used in the body to synthesize hemoglobin, which plays an important role in oxygen carrying capacity for athletes. Glycine also can be methylated in the body to become part of the one-carbon metabolic pathway. The one-carbon metabolic pathway is extremely important for the synthesis of anabolic, androgenic steroidal hormones as well as cortisone-like hormones. The methylation of glycine, that stimulates the one-carbon metabolic pathway is, in turn, stimulated by the presence of sufficient glycine. Glycine has also been reported to help trigger the release of oxygen to be used for energy in cell growth and replication, thereby helping to decrease tissue repair times, promoting faster recovery of the body after exercising. The dose of glycine has been carefully selected to accomplish all of the above. Glycine can be added in the form of L-glycine or peptide bonded glycine.

Cysteine is one of two sulfur containing amino acids commonly found in food sources. Of the two, methionine and cysteine, cysteine is the most metabolically valuable and the least common. Cysteine, in combination with glycine and glutamine, is a precursor to the manufacture of glutathione in the body. Glutathione is an important metabolic hormone that has been implicated in strengthening the immune system as well as increasing skeletal muscle mass. Cysteine also plays an important role in role in maintaining liver function and is currently being investigated for its role in maintaining blood sugar levels in hypoglycemics. Cysteine has been implicated in assisting with the storage and utilization of glycogen in muscle tissue. Cysteine has also been shown to have a synergistic metabolic function when combined with Vitamin B6. One can utilize L-cysteine or other, easier to handle forms, such as N-acetyl-cysteine, as the source for cysteine. N-acetyl-cysteine is preferred as the source for cysteine because studies have shown that N-acetyl-cysteine is more efficiently utilized by the body than L-cysteine after being consumed as part of a meal.

Leucine is a side branched chain amino acid that must be consumed daily through the diet in adequate quantities to meet the body's requirements. Leucine has been shown to assist in prevention of muscle wasting. Leucine has also been shown to have anabolic effects through prevention of muscle tissue breakdown and by stimulating protein synthesis in muscle tissue, as well as helping to prevent skeletal muscle injury. Leucine is also a gluconeogenic active agent. Leucine can act to assist in maintaining serum glucose levels for body energy during times of glucose depletion. Leucine, however, is only effective when taken in therapeutic dosage levels. We add leucine to the formula at therapeutic dosage levels. We prefer to use L-leucine in this formula, although one could also use leucine bonded to other amino acids.

Arginine is a vitally important non-essential amino acid. Arginine can be used by the body to synthesize ornithine. Ornithine has been shown to stimulate the release of human growth hormone. The release of growth hormone, in turn, promotes greater skeletal muscle mass. Arginine had also been shown to decrease tissue repair times. Decreased tissue repair times equate to faster recovery after exercise in sports nutrition. Arginine is provided in the form of L-arginine.

Vitamin B6 is involved in a wide variety of metabolic functions in the body. For example, Vitamin B6 is necessary for synthesis of more than 100 enzymes that assist in protein metabolism. Vitamin B6 has been shown to amplify the functions of peptides and amino acids in the body, demonstrating synergistic reactions with a number of amino acids. Vitamin B6 is also necessary for the manufacture of hemoglobin—that portion of blood responsible for the transport of oxygen throughout the body. Vitamin B6 has also been shown to actually increase the oxygen carrying capacity of hemoglobin, a very important factor in improving athletic performance. Vitamin B6 has also been shown to increase cell growth, including muscle tissue cells. The increased cell growth translates directly to faster muscle tissue repair times and faster recovery times after exercise. Vitamin B6 has been shown to assist in the conversion of stored carbohydrates to glucose for use in manufacturing energy required by a hard training body. Vitamin B6 is also a necessary agent for the body to manufacture carnitine, a nitrogen containing, short chain carboxylic acid. Carnitine is sometimes referred to as an amino acid but it is technically a cross between an amino acid and a vitamin. Studies have demonstrated that carnitine assists in the transport of fats from storage to the mitochondria where the fat is metabolized for energy. In sports nutrition; it is thought that the enhanced fat oxidation promoted by carnitine spares stored carbohydrates, allowing them to be utilized at more convenient times such as during hard training. Recently, researchers reported that carnitine may reduce lactic acid production in muscle tissue during aerobic exercise, thus leading to decreased recovery times after exercise. Many studies have shown that supplementing the diet with L-carnitine does not seem to produce the beneficial results listed above. Consequently, it appears that the only effective method for obtaining the benefits of carnitine is to provide the body with the tools necessary for the manufacture of carnitine. Of those tools, Vitamin B6 is the least common and most easily depleted. For our purposes, one can use any one of three forms of Vitamin B6—pyridoxine, pyridoxal, or pyridoxamine. The most common form of Vitamin B6, pyridoxine hydrochloride, is preferred.

The main purpose of exercise is to stimulate muscle tissue through contraction and extension of the muscle fibers. Each contraction and extension, if performed under stress, will cause some damage to the muscle tissue. This damage is then repaired after exercise is stopped and the repair cycle usually leads to formation of additional muscle tissue resulting in muscle growth. Muscle contraction and extension is accomplished via nerve stimulation of the muscle fibers. To ensure optimum operation of the nervous system, and therefore optimum contraction and extension of muscle fibers, some of the amino acids included in the formula, glutamine, taurine, glycine, and cysteine have neuromodulating properties. In addition to the neuromodulating properties of the amino acids, we have included sodium and potassium in the formula. Sodium and potassium are both widely known to be an integral part of nervous system transmissions. Sodium and potassium are also integral parts of the body's transport mechanism across cell membranes. It is the sodium active transport system and the potassium membrane transport system that helps to move the high glycemic sugar through the intestinal walls and the amino acids to muscle cells.

The last active ingredients included in the mixture are citric acid and sodium citrate. Both are contributors of citrate ion and they taste better when used in combination with each other, as is common practice in the food industry. Citrate is an important intermediate chemical in the ATP energy cycle known as the Krebs cycle or the Citric Acid cycle. The Krebs cycle is the metabolic process by which the body derives its required energy for all body functions, including repair of muscle tissue and muscle protein synthesis. It is hoped that inclusion of a citrate ion donating agent will stimulate the Krebs cycle process, thereby promoting more energy for the body to use for manufacturing additional muscle tissue. There is also some investigative evidence to support claims that free amino acids, such as are included in the mixture, are more efficiently absorbed and utilized by the body when they are consumed at a slightly acid pH.

For the purposes of consumer acceptance, flavors, color, and an artificial sweetener are added to the mixture. None of the flavors, the color, or the artificial sweetener are necessary ingredients, however.

As an example: We made a blend consisting of the following formula:

55.03% Glucose

15.86% Peptide Bonded Glutamine

11.90% Creatine Monohydrate

7.15% L-glycine

2.38% L-taurine

2.38% L-leucine

1.19% N-acetyl-cysteine

0.24% L-arginine

0.72% Citric Acid

0.71% Sodium Citrate

0.24% Sodium Chloride

0.24% Potassium Chloride

0.01% Pyridoxine Hydrochloride

2.41% Flavor, sweetener, and color

The ingredient levels were calculated so that each serving supplied 23 grams of high glycemic carbohydrate, 6.66 grams of peptide bonded glutamine, 5 grams of creatine, 3 grams of glycine, 1 gram of taurine, 1 gram of leucine, 0.5 grams of cysteine, 100 mg of arginine, 60 mg potassium, 50 mg sodium, and 200% of the USRDA for Vitamin B6. The pH of the total blend when dispersed into 16 ounces of water was 4.6.

A supply of 30 servings, 42 grams per serving, was provided to a number of athletes and to persons with stressful occupations, such as firemen. They were instructed to consume one serving each day dissolved in 16 ounces of cold water. They were further advised that the product would function best when it was consumed on an empty stomach—either one hour prior to eating a meal or 3 to 4 hours after eating a meal. Most people consumed the product first thing in the morning before they ate anything else for the day. A few of the testers consumed the product just prior to starting their exercise regime and others consumed the product immediately after exercising.

The feedback received from the test subjects indicated that the product was significantly anabolic. The product produced significant performance improvements in most test subjects within the first 48 to 72 hours. Bicycle racers reported that they were able to significantly reduce their racing times in specified length races. People who regularly worked out in gyms lifting weights reported that they were able to perform more lift repetitions with the same amount of weight than they had been able to before they started consuming the mixture. One subject reported that he was able to perform significantly higher numbers of sit-ups and push-ups after he started consuming the mixture. One subject reported losing 2 inches off of his waist and gaining an inch (at least) in his arm muscles in just weeks after he started consuming the mixture. A 55 year old gym owner reported that he was able to turn back the clock and felt like he was 10 years younger while working out after he started consuming the mixture. A professional bodybuilder reported that he was able to lose 2% of his body fat and gain 15 pounds of muscle tissue in just two weeks. He gave all credit for his transformation to the mixture. Almost all of the test subjects reported that they felt that their muscles had extra volume within days after they started using the mixture. Almost all of the test subjects reported that their exercise workouts were easier, requiring less effort than normal. Almost all of the test subjects reported that they were not as sore post-exercise as they would be normally. In general, the feedback suggests that the mixture allows an athlete to improve performance, perform tasks easier, recover from exercise faster, lose body fat, and gain muscle mass faster. These are the same benefits that are attributed to anabolic agents such as steroidal or cortisone-like hormones. The reports we received exceeded any benefits that would have been expected if one were to supplement their daily diet with each of the ingredients separately. The results our test subjects were realizing could only be achieved through a synergistic reaction of the ingredients so that they each amplified the functions of the other ingredients.

Of course, some of the formula ingredient usage levels could probably be modified without significantly altering the metabolic function of the blend. For instance, it is probably possible that the high glycemic carbohydrate content could be varied between 40% and 65% of the formula. The glutamine content could be varied from perhaps 10% of the formula to as much as 30% of the formula without sacrificing the end result. One could always increase the amount of creatine delivered (as much as triple) and still achieve the same result. One could increase the arginine content considerably without sacrificing functionality of the product. The other amino acids, however, are being delivered in just about the right amounts. The sodium, potassium, and citric acid/sodium citrate combination are also being delivered at the proper levels. One could probably quadruple the Vitamin B6 content without sacrificing the metabolic benefits of the product. We, however, subscribe to the theory that even though a small quantity of an ingredient is shown to be beneficial, it does not necessarily mean that larger doses would be equally as good or even better. We have tried to minimize the doses of each ingredients, providing efficacious amounts of each ingredient but not exceeding safe limits. Due to the synergism between the active ingredients that we have encountered, it is not necessary to exceed safe and healthy limits for any of the ingredients. It is doubtful that any extra benefits could be realized even if one were to significantly increase usage levels of many of the ingredients in the formula. Others could try to circumvent the protection of this technology by utilizing ingredient levels outside the range of the example above, but they would not be able to show an improvement to the technology by doing so. The ingredient levels we have chosen are the healthy and efficacious dose minimums and appear, from all apparent reports, to be sufficient to result in anabolic benefits for athletes. The synergistic reaction observed comes from the unique combination of all of the ingredients.

As various changes could be made in the above constructions without departing from the scope of the invention, it is intended that all matter contained in the above description or shown in the accompanying drawings shall be interpreted as illustrative and not in a limiting sense.

I claim:

1. A nutritional supplement comprising a source of high glycemic sugar and/or carbohydrate, a source of vitamin B6, and a source of amino acids; the source of amino acids including amino acids having neuromodulating properties and muscle cell volumizing properties.

2. The nutritional supplement of claim 1 further including electrolytic sodium and potassium.

3. The nutritional supplement of claim 1 further including a source of citrate ions.

4. The nutritional supplement of claim 3 wherein the source of citrate ions is chosen from the group consisting of citric acid, sodium citrate and combinations thereof.

5. The nutritional supplement of claim 1 wherein the source of high glycemic sugar and/or carbohydrate is chosen from the group consisting of sucrose, glucose, dextrose, fructose, complex carbohydrates, and combinations thereof.

6. The nutritional supplement of claim 5 wherein the complex carbohydrate is chosen from the group consisting of white flour, potato starch, rice starch, and combinations thereof.

7. The nutritional supplement of claim 1 wherein the amino acids include glutamine, taurine, creatine, cysteine, glycine, leucine, and arginine.

8. The nutritional supplement of claim 7 wherein the amino acids having neuromodulating properties is chosen from the group consisting of glutamine, taurine, glycine, and cysteine, and combinations thereof; and the amino acids having cell volumizing properties is chosen from the group consisting of creatine, taurine, and glycine, and combinations thereof.

9. The nutritional supplement of claim 7 wherein the creatine is chosen from the group consisting of N-acetyl-creatine, creatine phosphate, creatine citrate, creatine glycerol phosphate, and combinations thereof.

10. The nutritional supplement of claim 9 wherein the creatine is included in the supplement in an amount to provide about 5 gm of creatine per dose of the nutritional supplement.

11. The nutritional supplement of claim 7 wherein the glutamine is chose from the group consisting of L-glutamine, peptide bonded glutamine, and combinations thereof.

12. The nutritional supplement of claim 7 wherein the glycine is chosen from the group consisting of L-glycine, peptide bonded glycine, and combinations thereof.

13. The nutritional supplement of claim 7 wherein the cysteine is chosen from the group consisting of L-cysteine, N-acetyl-cysteine, and combinations thereof.

14. The nutritional supplement of claim 1 wherein vitamin B6 is chosen from the group consisting of pyridoxine, pyridoxal, pyridoxamine, and combinations thereof.

15. The nutritional supplement of claim 1 wherein the nutritional supplement is blended such that a single serving of the supplement comprises about 23 grams of high glycemic carbohydrate, about 6.66 grams of peptide bonded glutamine, about 5 grams of creatine, about 3 grams of glycine, about 1 gram of taurine, about 1 gram of leucine, about 0.5 grams of cysteine, about 100 mg of arginine, about 60 mg potassium, about 50 mg sodium, and about 200% of the USRDA for Vitamin B6.

16. The nutritional supplement of claim 15 wherein the pH of the total blend when dispersed into 16 ounces of water is about 4.6.

17. A nutritional supplement comprising a source of glucose, a source of glutamine, a source of creatine, a source of glycine, a source of taurine, a source of leucine, a source of cysteine, a source of arginine, a source of citrate, a source of sodium chloride, a source of potassium chloride, and a source of vitamin 6.

18. The nutritional supplement of claim 17 wherein the nutritional supplement is blended such that a single serving of the supplement comprises about 23 grams of high glycemic carbohydrate, about 6.66 grams of peptide bonded glutamine, about 5 grams of creatine, about 3 grams of glycine, about 1 gram of taurine, about 1 gram of leucine, about 0.5 grams of cysteine, about 100 mg of arginine, about 60 mg potassium, about 50 mg sodium, and about 200% of the USRDA for Vitamin B6.

19. The nutritional supplement of claim 18 wherein the pH of the total blend when dispersed into 16 ounces of water is about 4.6.

20. The nutritional supplement of claim 17 further including one or more of a flavoring agent, a sweetener and a coloring agent.

21. The nutritional supplement of claim 20 wherein the nutritional supplement comprises:
about 40% to about 65% glycemic carbohydrate or sugar;
about 10% to about 30% glutamine;
about 10% to about 30% creatine;
about 7.1% glycine;
about 2.4% taurine;
about 2.4% leucine;
about 1.2% cysteine;
about 0.2% arginine;
about 1.4% citrate;
about 0.2% sodium chloride;
about 0.2% potassium chloride;
about 0.01% to about 0.04% vitamin B6; and
about 2.4% flavor, sweetener, and color.

22. The nutritional supplement of claim 21 wherein the citrate comprises citric acid and sodium citrate; the citric acid comprising about 0.7% of the nutritional supplement and the sodium citrate comprising about 0.7% of the nutritional supplement.
